(19) United States
(12) Patent Application Publication
(10) Pub. No.: US 2017/0273710 A1
(43) Pub. Date: Sep. 28, 2017

(54) CATHETER COMPRISING A CUTTING ELEMENT

(71) Applicant: BALTON Sp. z o.o., Warszawa (PL)

(72) Inventors: Emil PLOWIECKI, Warszawa (PL); Leszek HURKALA, Warszawa (PL)

(21) Appl. No.: 15/514,014

(22) PCT Filed: Oct. 15, 2015
(86) PCT No.: PCT/PL2015/000167

§ 371 Date: Mar. 24, 2017

(30) Foreign Application Priority Data
Oct. 17, 2014 (PL) P.409824

(51) Int. Cl. A61B 17/3207; A61M 25/00

(52) U.S. Cl. CPC A61B 17/320725; A61M 25/0026; A61B 2017/320741

(57) ABSTRACT

A catheter including a cutting element and an insertion tube, wherein the springy resilient cutting element (1) consists of a sleeve (1a) and profiled longitudinal arms (1b) with sharp endings (1c), protruding from it and directed towards an atraumatic tip (7), wherein the element (1) is mounted on an assembly (2) of an inner tube.

The object of the present invention is a catheter comprising a cutting element, i.e. an element that can controllably cut (damage) internal areas of walls of a blood vessel.

More particularly, the invention relates to the field of medical devices introduced percutaneously into a blood vessel in order to perform the procedure of its mechanical or chemical and mechanical obliteration (ablation, closure).

Chronic venous insufficiency is a peripheral vascular disease observed in the half of the adult population and leads to the formation of varicose veins, so-called “spider veins”, venous inflammations, oedemas. Circulatory insufficiency in the lower extremities is a disease whose incidence increases with age, which results in an increasing demand for less invasive, non-surgical treatments to eliminate problems such as varicose veins of the lower extremities.

Venous stasis is a disorder caused by abnormal blood flow from the lower extremities to the heart. Contrary to appearances, not only the heart is responsible for ensuring the proper blood circulation in the organism. The work of the leg muscles (muscle pump) is responsible for returning blood to the heart. In a healthy body, muscles allow blood to return to the heart through the venous valve system. When lesions are observed, the “muscle pump” in the area of the extremities fails to work and/or the valve system does not sufficiently support the return of blood to the heart.

Methods of treating venous insufficiency (varicose veins) are limited. Currently used methods are based on stripping or ligation of insufficient venous sections, vein ablation using a laser or by the application of ultrasound, or steam, as well as through chemical obliteration by local injection of chemical substances that cause irritation to the inner walls of veins and, as a result, their coalescence (closing).

These methods have certain disadvantages, for example they may cause skin inflammations, vascular perforations, skin lesions or secondary venous patency (recanalisation). Furthermore, there are observed side effects related to the effects of the mentioned factors on blood cells and sometimes on adjacent, healthy tissues, including blood vessels, e.g. in the laser method causes overheating of the area surrounding the place of procedure, and also possible is the formation of clots that cause blockages in other areas of the patient's body.

The present invention solves many of the problems indicated above by providing a simple and effective mechanical method of closing veins.

There is known the phenomenon that mechanical irritation to the inner walls of the vein causes its contractions. Sometimes, not very skilful use of intravascular medical equipment may cause contraction of the vessel. In the area where the vessel endothelium is mechanically damaged, cut, scratched or injured, a self-acting reaction of contraction (tightening) of the vessel is caused.

Within the present invention, a catheter provided with technical means for mechanical irritation of the vessels is proposed. Such a device allows solving many of the problems signalled above. Moreover, the developed catheter allows simultaneous support of mechanical obliteration by chemical obliteration, i.e. the catheter is provided with a channel for the administration of active substances. At the same time, the proposed solution helps to minimise the size of the catheter, which allows it to operate in narrow, deformed, affected vessels.

The object of the invention is the catheter comprising the cutting element and the insertion tube, characterised in that the elastic (springy resilient) cutting element comprises the sleeve and longitudinally profiled arms with sharp endings coming out of it, directed towards the atraumatic tip, wherein the cutting element is mounted on the assembly of the inner tube.

The assembly of the inner tube comprises the distal tube of a smaller diameter and the body tube of a greater diameter, forming the proximal portion of the assembly, wherein the cutting element is mounted on the distal tube.

The sleeve has a diameter equal to the diameter of the body tube of the assembly of the inner tube and this sleeve, from its proximal side, is immediately adjacent to the body tube.

The cutting element comprises 3 to 10 arms, which are preferably arc-shaped outward with respect to the axis of the catheter, and in the released state, in the vicinity of the sleeve, these arms are arranged at an acute angle in relation to the axis of the catheter.

The sharp endings of the cutting element are bent away from the arms outwardly with respect to the axis of the catheter, and these endings are conically pointed or form an elongated blade.

Along the catheter, the channel for the guidewire is led.

The catheter comprises a channel for fluid supply.

The cutting element is made of a single tube segment.

The insertion tube has an outer diameter of less than 2.2 mm.

The degree of release and unfolding of the arms of the cutting element is adjusted with the position of the distal edge of the insertion tube in relation to this element.

The cutting element has a shape adapted for repeated and multiple insertion of it entirely within the insertion tube, which in this position is in contact with the tip.

The object of the present invention in an exemplary embodiment is illustrated in the drawing, in which

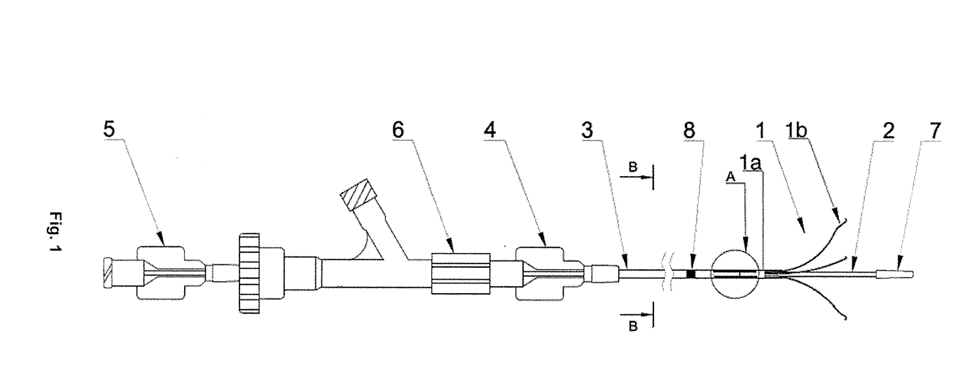

FIG. 1 shows a general view of the catheter with the cutting element completely released from the insertion tube, with the detail “A” and the plane of the cross section B-B also marked,

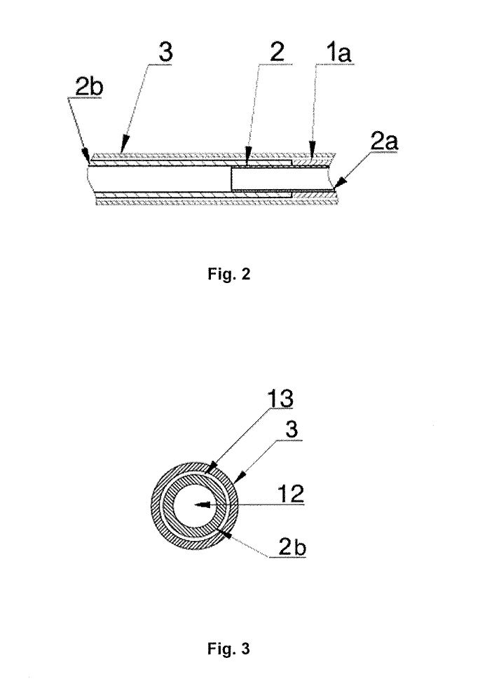

FIG. 2 shows an enlarged detail “A” marked in FIG. 1 showing the point of connection of the body tube and the distal tube, as well as the place of mounting the sleeve of the cutting element, and

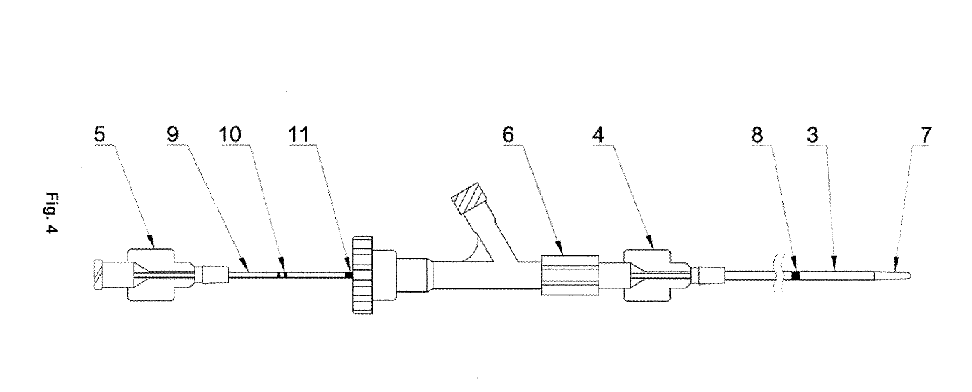

FIG. 3 shows an enlarged B-B cross-sectional view of the catheter in the place marked on the FIG. 1,

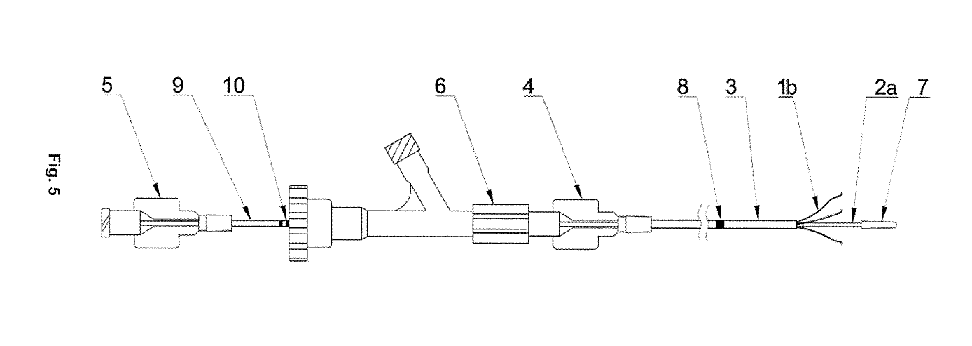

FIG. 4 shows the catheter of FIG. 1 in the closed position, with the cutting element hidden under the insertion tube,

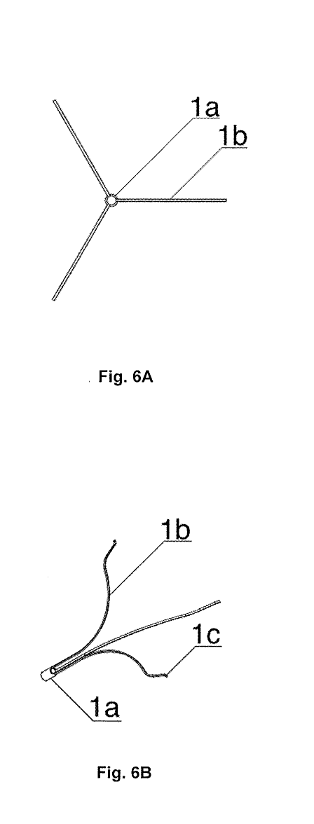

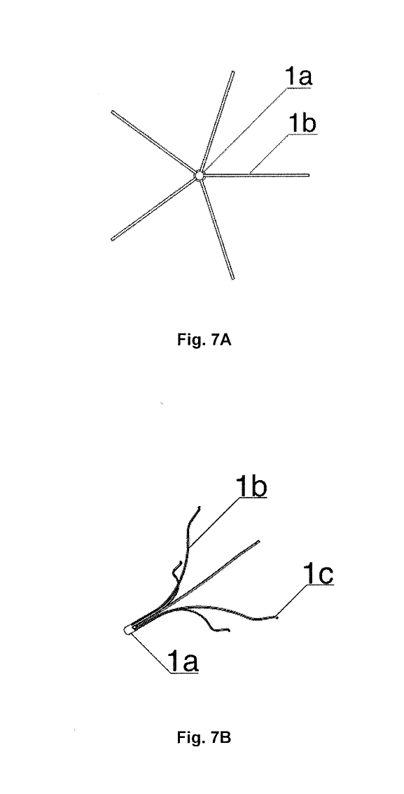

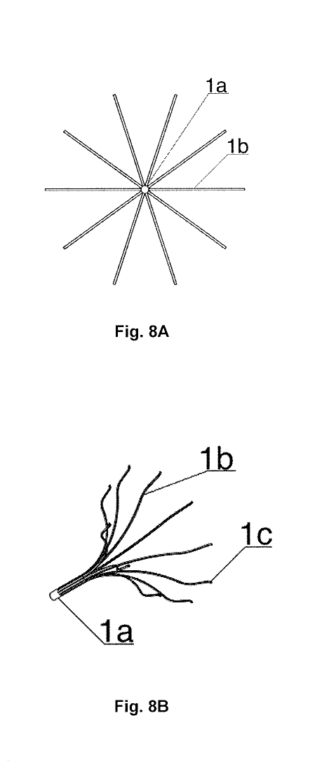

FIG. 5 shows the catheter of FIGS. 1 and 4 in an intermediate position, with a partially extended cutting element, which is partially overlapped by the insertion tube, FIGS. 6A and 6B, 7A and 7B, and 8A and 8B show an enlarged structure of the cutting element with, respectively, three, five and ten arms, wherein

FIGS. 6A, 7A and 8A show a view of the cutting elements exploded (open) from the side of their sharp ends, and FIGS. 6B, 7B and 8B show a general view of these cutting elements in an enlargement.

The catheter according to the present invention comprises a springy resilient (elastic) cutting element that is mounted on the distal tube 2a of the assembly 2 of the inner tube, and comprises, successively, viewed from the proximal (the operator's) side, the sleeve 1a and the arc-curved longitudinal arms 1b with the properly formed and bent outwards sharpened endings 1c, connected thereto at its distal edge and facing towards the atraumatic tip 7. The arms 1b form a band of identical elements (branches of the cutting element protruding from the axis of the catheter). It is possible to profile the arms 1b in any way in order to achieve the effect of irritation of the vessel wall by the sharp endings 1c. The assembly 2 of the inner tube consists of two concentrically arranged tubes, the distal tube 2a and the body tube 2b of a larger diameter, which is the proximal portion of the assembly 2 of the inner tube. The distal tube 2a is adapted in its diameter to the body tube 2b, so that it is possible to contiguously mount the tubes one in another at the point of their connection (shown in FIG. 2). The body tube 2b supports the tube 2a with a smaller diameter, located further, in the distal portion of the catheter. The sleeve 1a of the cutting element 1 has a diameter equal (or nearly equal) to the diameter of the body tube 2b. The described mutual arrangement of the tubes 2a and 2b and the sleeve 1a is shown in FIG. 2. The insertion tube 3, with a diameter larger than the tube 2a and the sleeve 1a, covers both these elements, together with the arms 1b and the endings 1c of the cutting element, and therefore the cutting element 1 without obstacles can be repeatedly released and retracted by sliding it within the tube 3. The insertion tube 3 has preferably an outer diameter of less than 2.2 mm. Preferably, the sleeve 1a is mounted on the distal tube 2a in such a manner that the proximal edge of the sleeve 1a is in direct contact with the distal edge of the body tube 2b.

Number of the arms 1b of the cutting element is in the range of 3 to 10, e.g. the cutting element can comprise 3, 4, 5, 6 arms 1b.

Preferably, the arms 1b in relation to the axis of the sleeve 1a and the axis of the catheter are arranged symmetrically e.g. on the circumference of the sleeve at every 120° (3 arms) or every 90° (4 arms), etc., as shown demonstratively in FIGS. 6A, 7A and 8A. The arms 1b have the form of branches extending from the sleeve 1a and can have a circular cross-section (particularly when formed from wires attached to the sleeve) or approximately trapezoidal cross-section (when formed by cutting out the arms and the sleeve from one section of metal tube). The arms 1b are formed so that when releasing the element 1 from the catheter they radially extend outward, away from the assembly 2 of the inner tube towards the walls of the blood vessel. The arms 1b after full extension of the cutting element (releasing it from the insertion tube 3) are arranged in a section adjacent to the sleeve at an acute angle in relation to the axis of the catheter, e.g. at an angle of 60° or 45°. The arms 1b of the end portion, remote from the sleeve 1a, are profiled (bent) arcuately, so that the sharp endings 1c are adjacent to the walls of the vessel. The arms 1b can also be formed as a line similar to the letter “S”, or in a yet different way, however, the endings 1c are always most remote from the axis of the catheter. Preferably, the sharp endings 1c are bent away from the arms 1b outwardly with respect to the axis of the catheter, i.e. they are more strongly curved outwardly than the line of curvature of the arms 1b. The endings 1c can be conically pointed or can create slightly elongated blade by flattening the end sections of the arms 1b.

The cutting element 1 can be completely cut out from the section of the tube of metal or other material and then the beginning of such a tube is not treated (the sleeve remains) and in the remaining portion of the tube cutting is carried out to obtain a desired number of arms, therefore 3 to 10 arms are cut in the tube. It is also possible to prepare the cutting element 1 by mounting previously prepared single arms 1b to the section of the tube (the sleeve). These arms are mounted to the sleeve in such manner that the diameter of the retracted cutting element 1 along its length does not exceed the diameter of the sleeve 1a, when the element 1 is retracted in the insertion tube 3. As a result, the catheter can have a minimum diameter limited only with the diameter of the sleeve 1a, increased by the addition of the insertion tube 3. Between the insertion tube 3 and the assembly 2 of the inner tube the space is maintained constituting the channel 13 for fluid supply, for example of sclerotisation chemicals (including foams), pharmacologically active agents, saline solution, etc. Inside the assembly 2 of the inner tube, along the entire catheter, the channel 12 for the guidewire was led, preferably for the guidewire of the dimension 0.035″. The guidewire provides stable positioning of the catheter during the procedure.

In its proximal portion, the catheter comprises known in the art elements accessible for the operator, enabling control of the device. Subsequently, from the proximal side, the catheter is provided with the cap 5 of the assembly of the inner tube, from which the cuff 9 of the assembly of the inner tube is led, next the “Y” cap 6 and the cap 4 of the insertion tube. The cap 4 and the “Y” cap 6 are used together to control the insertion tube.

On the distal tube 2a, at its end, after the cutting element 1, there is mounted the atraumatic soft tip 7 enabling atraumatic insertion of the catheter into the vessel. The catheter can preferably comprise markers visible through ultrasound and/or X-ray imaging, enabling operation of the device during procedure. On the insertion tube 3, at its distal portion, there is provided the marker 8, and on the body tube 2b, at its proximal side, there is provided the first marker 10 followed by the second marker 11. Along the entire catheter there is led the internal channel 12 for the guidewire, shown as the middle space in FIG. 3.

The catheter is inserted into the blood vessel within the guidewire previously positioned in said vessel, through a typical shrink in the position where the insertion tube 3 covers the entire retracted cutting element 1 (the arms 1b and the endings 1c then adhere to the distal tube 2a). The insertion tube 3 comes then to the soft tip 7—the catheter is closed. Full extension of the cutting element 1 occurs when pushing the cap 5 all the way to the “Y” cap 6.

To lock the position of the assembly 2 of the inner tube in relation to the insertion tube 3 one needs to tighten the nut on the “Y” cap 6. Tightening the nut seals the space between the insertion tube 3 and the assembly 2 of the inner tube, and thus venting of the channel 13 shown in FIG. 3 is possible. At the distal portion of the insertion tube 3 there is applied the marker 8, which after extension off the shrink during procedure means that the cutting element 1 is now pushed all the way to the shrink. At the proximal portion of the assembly 2 of the inner tube there are applied two markers. The marker 11 means complete retraction of the cutting element 1 in the catheter, the marker 10 means partial extension of the cutting element 1.

FIG. 5 shows the cutting element 1 partially extended (released) from the insertion tube 3. In such a position, the cutting element can also fulfill its function, i.e. the sharp ends 1c are in contact with the inner wall of the vessel and cause its longitudinal incisions when moving the catheter. The described construction of the catheter thus does not require the use of a completely extended cutting element 1 during the procedure. Depending on the structure, size and shape of the affected vessel, the operator can smoothly grade the folding of the arms 1b, i.e. adjust the diameter of the extended cutting element to the diameter of the vessel by adjusting the position of the insertion tube 3 in relation to the element 1. The “Y” cap 6 enables locking the cutting element at a predetermined position.

The above described construction of the catheter allows smooth and fast multiple repeats of the procedure of incision of the vessel, i.e. its mechanical sclerotisation/obliteration. The cutting element 1 is susceptible to reproducible, multiple insertions of it entirely within insertion tube 3. After the insertion of the catheter and release of the cutting element 1 from the insertion tube 3, it is moved with a uniform motion in the reverse direction (back to the operator), which causes longitudinal incision (irritation, scratch) of the vessel endothelium along the required length of the sclerotised vein. Then the cutting element 1 can be retracted in the insertion tube 3 (still intravascularly), moved in this closed position again to a remote (from the operator) portion of the vessel, and then released again, so that the process of incision of the vessel is repeated on the same or another portion of the vessel. As a result of irritation of the vessel there are almost immediately caused contractions of the vessel and its closure.

The procedure using the catheter according to the present invention can be carried out also as a mechanical and chemical sclerotisation. In such a case the operations described above are repeated, but through the side channel of the “Y” cap 6 there is additionally administered a dose of sclerotisation agent in the form of a liquid solution or a foam prepared using the Tessari method. The catherer is pulled (withdrawn) with a uniform motion, cutting the endothelium of the vein with the cutting elements 1 while injecting sclerotisation agent. As before, the operation should be performed over the entire length of sclerotised vein. Mechanical and chemical sclerotisation of the same vein can be performed repeatedly.

### The Marks on the Drawings:

1 cutting element
1a sleeve of the cutting element
1b arm/arms of the cutting element
1c sharp ending of the arm
2 assembly of the inner tube
2a distal tube
2b body tube
3 insertion tube
4 cap of the insertion tube
5 cap of the assembly of the inner tube
6 “Y” cap
7 soft tip
8 marker of the insertion tube
9 cuff of the assembly of the inner tube
10 first marker of the body tube
11 second marker of the body tube
12 channel for the guidewire
13 channel for fluid supply

1. A catheter comprising a cutting element and an insertion tube, wherein the springy resilient cutting element consists of a sleeve and profiled longitudinal arms with sharp endings, protruding from it and directed towards an atraumatic tip, wherein the element is mounted on an assembly of an inner tube.

2. The catheter according to claim 1, wherein the assembly of the inner tube comprises a distal tube with a smaller diameter and a body tube with a larger diameter, constituting the proximal portion of the assembly, wherein the cutting element is mounted on the distal tube.

3. The catheter according to claim 1, wherein the sleeve has a diameter equal to the diameter of a body tube of the assembly of the inner tube.

4. The catheter according to claim 1, wherein the sleeve at its proximal side is immediately adjacent to a body tube.

5. The catheter according to claim 1, wherein the cutting element comprises 3 to 10 arms.

6. The catheter according to claim 1, wherein the cutting element has arms arc-shaped outwardly with respect to the axis of the catheter, and in the released state, in the vicinity of the sleeve, the arms are arranged at an acute angle in relation to the axis of the catheter.

7. The catheter according to claim 1, wherein sharp endings are bent away from the arms outwardly in relation to the axis of the catheter, and these endings are conically pointed or form an elongated blade.

8. The catheter according to claim 1, wherein along the catheter there is led a channel of the guidewire.

9. The catheter according to claim 1, wherein the catheter comprises a channel for fluid supply.

10. The catheter according to claim 1, wherein the cutting element is made from a single tube segment.

11. The catheter according to claim 1, wherein the insertion tube has an outer diameter of less than 2.2 mm.

12. The catheter according to claim 1, wherein the degree of release and unfolding of the arms of the cutting element is adjusted with the position of the distal edge of the insertion tube with respect to this element.

13. The catheter according to claim 1, wherein the cutting element has a shape adapted for repeated and multiple insertion of it entirely within the insertion tube, which in this position is in contact with a tip.
